(19) United States
(12) Patent Application Publication
(10) Pub. No.: US 2019/0008837 A1
(43) Pub. Date: Jan. 10, 2019

(54) OPHTHALMIC COMPOSITIONS AND ASSOCIATED METHODS

(71) Applicant: University of Utah Research Foundation, Salt Lake City, UT (US)

(72) Inventors: Balamurali K. Ambati, Sandy, UT (US); Santosh Muddana, Salt Lake City, UT (US); Xiaohaui Zhang, Salt Lake City, UT (US)

(73) Assignee: University of Utah Research Foundation, Salt Lake City, UT (US)

(21) Appl. No.: 15/844,506

(22) Filed: Dec. 15, 2017

Related U.S. Application Data
(60) Provisional application No. 62/434,963, filed on Dec. 15, 2016.

(51) Int. Cl. A61K 31/437; A61K 9/127; A61K 45/06; A61K 9/00

(52) U.S. Cl. CPC A61K 31/437; A61K 9/0048; A61K 45/06; A61K 9/127

(57) ABSTRACT

Ophthalmic formulations and compositions employing pergolide to treat an ophthalmic or ocular condition are disclosed and described.

### PRIORITY DATA

This application claims the benefit of U.S. Provisional Application Ser. No. 62/434,963, filed on Dec. 15, 2016, which is incorporated herein by reference.

### BACKGROUND

Neurotrophic keratopathy (NK) is a devastating corneal condition where the ophthalmic branch of the trigeminal nerve is damaged due to infection, surgery, tumors, and/or trauma. NK can lead to corneal scarring, melting, and/or blindness. Approximately 160,000 people in the United States suffer from this condition and treatment options are limited. Traditional care has been to protect the cornea through suturing the eyelids closed.

### BRIEF DESCRIPTION OF THE DRAWINGS

FIG. 1A shows a cornea flatmount image of a normal cornea;

FIG. 1B shows a cornea flatmount image of a blank treated cornea;

FIG. 1C shows a cornea flatmount image of a pergolide treated cornea;

FIG. 1D shows a cornea flatmount image of a cabergoline treated cornea.

FIG. 2A shows a cornea flatmount image of a blank treated cornea;

FIG. 2B shows a cornea flatmount image of a cabergoline treated cornea; and

FIG. 2C shows a cornea flatmount image of a pergolide treated cornea.

FIG. 3A illustrates a bar chart depicting increased nerve growth factor (NGF) expression upon treatment with pergolide.

FIG. 3B illustrates western blot data at 24 hours after treatment with pergolide.

FIG. 4 illustrates a bar chart depicting increased GDNF expression upon treatment with pergolide.

FIG. 5A shows a cornea flatmount image of a normal cornea;

FIG. 5B shows a cornea flatmount image of a blank treated cornea—one week;

FIG. 5C shows a cornea flatmount image of a pergolide treated cornea—one week;

FIG. 5D shows a cornea flatmount image of a blank treated cornea—two weeks; and

FIG. 5E shows a cornea flatmount image of a pergolide treated cornea—two weeks.

FIG. 5F shows images of blank v. pergolide cornea healing at various time points.

FIG. 6 illustrates a bar chart depicting average cornea nerve areas in a cornea scratch model after one and two weeks of treatment with pergolide.

FIG. 7A shows a whole flatmount of a blank treated cornea;

FIG. 7B shows a whole flatmount of an enlarged flatmount section to illustrate nerve regeneration of the blank treated cornea;

FIG. 7C shows a whole flatmount of a pergolide treated cornea; and

FIG. 7D shows an enlarged flatmount section to illustrate nerve regeneration of the pergolide treated cornea.

FIG. 8 illustrates a graph of cornea nerve area after treatment 3 times per day for 5 days with blank or pergolide eye drops using a cornea scratch model. N=3 in each group. T test was used to analyze statistic difference. P=0.013

FIG. 9 illustrates a graph of cornea nerve area after treatment 3 times day for 1 week with blank or pergolide eye drops using a cornea scratch model. N=6 in each group. One-way ANOVA was used to analyze statistical difference.

FIG. 10A shows a flatmount of a blank treated cornea;

FIG. 10B shows a flatmount of a 300 μg pergolide dose-treated cornea; and

FIG. 10C shows a flatmount of a 600 μg pergolide dose-treated cornea.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this written description, the singular forms “a,” “an” and “the” include express support for plural referents unless the context clearly dictates otherwise. Thus, for example, reference to “a cell” includes a plurality of such cells.

As used herein, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The term “coupled,” as used herein, is defined as directly or indirectly connected in a chemical, mechanical, electrical or nonelectrical manner. “Directly coupled” objets or elements are in physical contact with one another.

Objects described herein as being “adjacent to” each other may be in physical contact with each other, in close proximity to each other, or in the same general region or area as each other, as appropriate for the context in which the phrase is used.

Occurrences of the phrase “in one embodiment,” or “in one aspect,” herein do not necessarily all refer to the same embodiment or aspect.

As used herein, the terms “therapeutic agent,” “active agent,” “drug,” and the like can be used interchangeably and refer to agent that can have a beneficial or positive physiologic effect on a subject when administered to the subject in an appropriate or effective amount.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician, veterinarian, or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986). It is also noted that, in some cases, a therapeutically effective amount can be achieved over a period of time and/or after a number of doses have been administered, such as in a dosing regimen.

As used herein, a “dosing regimen” or “regimen” such as “treatment dosing regimen,” or a “prophylactic dosing regimen” refers to how, when, how much, and for how long a dose of an active agent or composition can or should be administered to a subject in order to achieve an intended treatment or effect.

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, “pergolide” refers to a compound having the formula:

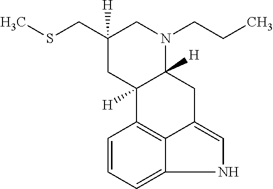 [SMILES: CCCN1C[C@H](CSC)C[C@@H]2c3cccc4[nH]cc(c34)C[C@H]21]

or otherwise having the IUPAC name, (6aR,9R,10aR)-9-(methylthiomethyl)-7-propyl-4,6,6a,7,8,9,10,10a-octahydroindolo[4,3-fg]quinolone, as well as salts, (e.g. pergolide mesylate, isomers, prodrugs and metabolites thereof. Pergolide acts as an agonist of dopamine D₂ and D₁ and serotonin 5-HT_{1A}, 5-HT_{1B}, 5-HT_{2A}, 5-HT_{2B}, and 5-HT_{2C} receptors. Examples of various specific pergolide forms and formulations can be found in U.S. Pat. Nos. 4,797,405 and 5,114,948, each of which are incorporated herein by reference.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (i.e. solution), or gas. Furthermore, the term “dosage form” can include one or more formulation(s) or composition(s) provided in a format for administration to a subject. For example, an injectable dosage form would be a formulation or composition prepared in a manner that is suitable for administration via injection.

As used herein, a “subject” refers to an animal. In one aspect the animal may be a mammal. In another aspect, the mammal may be a human.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

The cornea is the outermost layer of the eye and acts as a barrier against a variety of particles and other substances that can harm the eye. Further, the cornea works together with the lens of the eye to focus light on the retina, which translates light into vision. Thus, trauma, diseases, and disorders of the cornea can negatively impact vision and even be devastating to those suffering from such conditions. In some cases, these devastating conditions can lead to substantial or complete blindness.

One such condition is neurotrophic keratopathy (NK). NK is a devastating corneal condition where the ophthalmic branch of the trigeminal nerve is damaged due to infection, surgery, tumors, and/or trauma. NK can lead to corneal scarring, melting, and/or blindness. Approximately 160,000 people in the United States suffer from this condition. However, there are very limited treatment options for those suffering from this devastating condition. In fact, traditional care has been to merely attempt to protect the cornea through suturing the eyelids closed. Therefore, there is a substantial need for improved treatment options for those suffering from this and other ocular conditions.

Accordingly, the current disclosure describes ophthalmic compositions or dosage forms that can be used to treat a variety of ocular conditions. Generally, the ophthalmic compositions or dosage forms can include a therapeutically effective amount of pergolide and a pharmaceutically acceptable carrier.

The compositions and dosage forms can be formulated in a variety of ways to deliver pergolide to an eye of a subject. Non-limiting examples can include solutions, suspensions, gels, thin films, depots, and the like. In one specific example, the composition can be formulated as a topical ophthalmic drop, gel, or film. In another example, the composition can be formulated as an injectable composition, such as a composition formulated for intravitreal injection or subconjunctival injection, for example.

A therapeutically effective amount of pergolide can provide a number of benefits when administered (e.g. locally) to the eye. For example, a therapeutically effective amount of pergolide can increase nerve growth factor (NGF) synthesis and secretion in an eye as compared to a time prior to treatment. In some examples, a therapeutically effective amount of pregolide can increase glial cell line-derived nerotrophic factor (GDNF) synthesis in an eye as compared to the eye prior to treatment. In some additional examples, the therapeutically effective amount of pergolide can increase a rate of healing in an ocular tissue, such as the cornea, as compared to a healing rate in an untreated eye.

In some embodiments, a therapeutically effective amount of pergolide can be from 0.00005 mg/μl (0.05 μg/μl) to 0.05 mg/μl (50 μg/μl). In other embodiments, a therapeutically effective amount can be from about 0.0001 mg/μl (0.1 μg/μl) to 0.025 mg/μl (25 μg/μl). In yet other embodiments, a therapeutically effective amount can be from about 0.0001 mg/μl (0.1 μg/μl) to 0.015 mg/μl (15 μg/μl).

In some examples, the composition or dosage form can include an active agent in addition to pergolide. Non-limiting examples can include cabergoline, tacrolimus, glycyl-L-histidyl-L-lysine (GHK), nerve growth factor (NGF), the like, or a combination thereof.

The composition or dosage form can include a variety of pharmaceutically acceptable carriers. For example, a pharmaceutically acceptable carrier can include a thickening or gelling agent, a solubilizing agent, a tonicity agent, a pH adjuster, a preservative, an antioxidant, water, the like, or combinations thereof.

Non-limiting examples of thickening or gelling agents can include glycerol, propylene glycol, polyethylene glycol, polyvinyl alcohol, cellulose derivatives, such as methyl cellulose, carboxymethyl cellulose, hydroxypropyl cellulose, and the like, ethylvinyl alcohol, xanthan gum, guar gum, polyvinyl pyrrolidone, polyacrylic acid, hyaluronic acid, poloxamers, dextrans, the like, salts thereof, or combinations thereof. Thickening agents can be present in the pharmaceutically acceptable carrier in various amounts. In one aspect, the thickening agent can have a concentration in the carrier of from about 0.01 wt %, about 0.05 wt %, about 0.1 wt %, or about 0.5 wt % to about 2 wt %, about 8 wt %, about 15 wt %, or about 30 wt %.

Non-limiting examples of solubilizing agents can include phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, BSS Plus, Ringer's lactate solution, normal saline (i.e. 0.9% saline), ½ normal saline, glycerol, propylene glycol, polyethylene glycol, poloxamers and a variety of other surfactants, emulsifiers, and/or dispersing agents, the like, or combinations thereof. Solubilizing agents can be present in the pharmaceutically acceptable carrier in various amounts. In one aspect, the solubilizing agent can have a concentration in the carrier of from about 20 wt %, about 30 wt %, about 40 wt %, or about 50 wt % to about 80 wt %, about 90 wt %, about 95 wt %, about 97 wt %, or about 99 wt %.

Non-limiting examples of tonicity agents can include the solubilizing agents previously listed, as well as sodium chloride, potassium chloride, calcium chloride, magnesium chloride, mannitol, sorbitol, dextrose, glycerin, propylene glycol, ethanol, trehalose, the like, or combinations thereof. The tonicity agent can be used to provide an appropriate tonicity of the formulation. In one aspect, the tonicity of the formulation is from about 250 to about 350 milliosmoles/liter (mOsm/L). In another aspect, the tonicity of the formulation is from about 270 to about 330 mOsm/L. Tonicity agents can be present in the pharmaceutically acceptable carrier in various amounts. In one aspect, the tonicity agent can have a concentration in the carrier of from about 0.5 wt %, about 1 wt %, about 5 wt %, about 10 wt %, or about 20 wt % to about 30 wt %, about 40 wt %, about 50 wt %, or about 60 wt %.

Non-limiting examples of pH adjusters can include a number of organic and/or inorganic acids, bases, or combinations thereof, such as hydrochloric acid, phosphoric acid, citric acid, ascorbic acid, sodium hydroxide, potassium hydroxide, calcium hydroxide, the like, or combinations thereof. The pH adjusters can be used to provide an appropriate pH for the formulation. In one aspect, the pH can be from about 5 to about 8. In another aspect, the pH can be from about 6.0 to about 7.4. pH adjusters can be present in the pharmaceutically acceptable carrier in various amounts. In one aspect, the pH adjuster can have a concentration in the carrier of from about 0.01 wt %, about 0.05 wt %, about 0.1 wt %, or about 0.5 wt % to about 1 wt %, about 2 wt %, about 5 wt %, or about 10 wt %.

Non-limiting examples of preservatives can include benzalkonium chloride (BAK), cetrimonium, sodium perborate, ethylenediaminetetraaceticacid (EDTA) and its various salt forms, chlorobutanol, the like, or combinations thereof. Preservatives can be present in the pharmaceutically acceptable carrier in various amounts. In one aspect, the preservative can have a concentration in the carrier of from about 0.001 wt %, about 0.005 wt %, about 0.01 wt %, or about 0.05 wt % to about 0.1 wt %, about 0.25 wt %, about 0.5 wt %, or about 1 wt %.

Non-limiting examples of antioxidants can include vitamin E, carnosine, N-acetylcarnosine, pyruvate, resveratrol, astaxanthin, glutathione, cysteine, cysteine ascorbate, the like, or combinations thereof. Antioxidants can be present in the pharmaceutically acceptable carrier in various amounts. In one aspect, the antioxidant can have a concentration in the carrier of from about 0.01 wt %, about 0.05 wt %, about 0.1 wt %, or about 0.5 wt % to about 1 wt %, about 2 wt %, about 5 wt %, or about 10 wt %.

In another example, the pharmaceutically acceptable carrier can include liposomes. In some examples, at least a portion of the therapeutically effective amount of pergolide can be encapsulated or entrapped within a liposome. A number of compounds can be used to form the liposomes. Non-limiting examples can include a phospholipid, cholesterol, a lipid-conjugated hydrophilic polymer, or combinations thereof. In one specific example, the liposomes can be formed using cholesterol and a phospholipid, such as 1,2-distearoyl-sn-glycero-3-phosphocholine, for example.

Another embodiment includes a system for treating an ophthalmic disease or disorder in a subject. The system can include a composition or dosage form as described herein and a container. The therapeutic composition or dosage form can be held in or stored in the container as a pre-mixed composition that is ready to administer without further dilution. In some embodiments, a single container may provide a volume that is adequate for a single dose. In some embodiments, a single container may provide a volume that is adequate for a plurality of doses.

A number of suitable containers can be used. In one aspect, the container can be an amber-colored container. The container can be made of glass, polypropylene, polyethylene, polyvinylchloride, other suitable material, and combinations thereof. In some specific examples, the container can be adapted to dispense the composition in a dropwise manner. In some examples, the drop size can range from 10 μL or 20 μL to 50 μL or 60 μL.

Further included in a system can be an administration mechanism, such as a syringe or other mechanism. In additional embodiments, suitable packaging can be used to provide the composition, container, and instructions for the use thereof, and optionally an administration mechanism in a single integrated system.

The compositions or dosage forms described herein can also be employed in a method of treating an ophthalmic condition. Such a method can include administering a therapeutically effective amount of pergolide to an eye of a subject during a treatment period or in an effective dosage regimen. The method can be used to treat a number of ocular conditions. It is also noted, that while pergolide can be very effective for treating certain corneal conditions such as NK, it can also be used to treat a number of non-corneal conditions. Non-limiting examples of diseases or conditions for which the method can be effective include neurotrophic keratopathy (NK), post laser-assisted in situ keratomileusis (LASIK) nerve damage, post radial keratotomy (RK) nerve damage, post penetrating keratoplasty (PK) nerve damage, dry eye, dry age-related macular degeneration (AMD), glaucoma, diabetic retinopathy, retinal degeneration, the like, or combinations thereof.

The therapeutically effective amount of pergolide can be administered in a number of ways. In some examples, administration can be performed via intravitreal injection, subconjunctival injection, topical administration, or a combination thereof. In some specific examples, pergolide can be administered as a topical eye drop or gel.

In one example, the composition or dosage form can be administered at from 1 to 4 time points per day per eye in need thereof. In some examples, the dosage amount at each time point can be from about 10 μl or 20 μl to about 50 μl or 60 μl. In some examples, each dosage (e.g. drop) can include from about 0.005 mg, about 0.01 mg, or about 0.1 mg to about 0.7 mg, about 1 mg, or about 3 mg pergolide.

Generally, the methods described herein can up-regulate expression of NGF within 48 hours, 24 hours, or 18 hours as compared to expression of NGF prior to administration of pergolide. Further, the methods described herein can up-regulate expression of GDNF within 48 hours, 24 hours, or 18 hours as compared to expression of GDNF prior to administration of pergolide.

### EXAMPLES

### Example 1

### Up-Regulation of NGF and GDNF

Nine mice were divided into three treatment groups having three mice each. The corneas of each of the mice in each of the groups were scratched. The control group was administered a standard blank (e.g. pacebo) composition. The first treatment group received pergolide. The second treatment group received cabergoline. Each of the eye drops was applied 3 times per day for 5 days. The corneas were harvested on the 7th day. The corneas were then fixed in acetone for 1 hour at room temperature and then blocked in 3% BSA for 1 hour. The cornea flatmounts were stained with primary antibody, anti-beta III tubulin antibody (rabbit polyclonal, ab18207) at 1:200 in 3% BSA blocking buffer overnight at 4° C. After washing 3 times for 10 minutes, each of the cornea flatmounts were incubated with secondary antibody (goat anti rabbit, Invitrogen Alexa Fluor 555) at 1:200 in 3% BSA for 1 hour at room temperature. The flatmounts were again washed 3 times for 10 minutes each. Images of the cornea neuron fibers were taken and analyzed under EVOS microscopy.

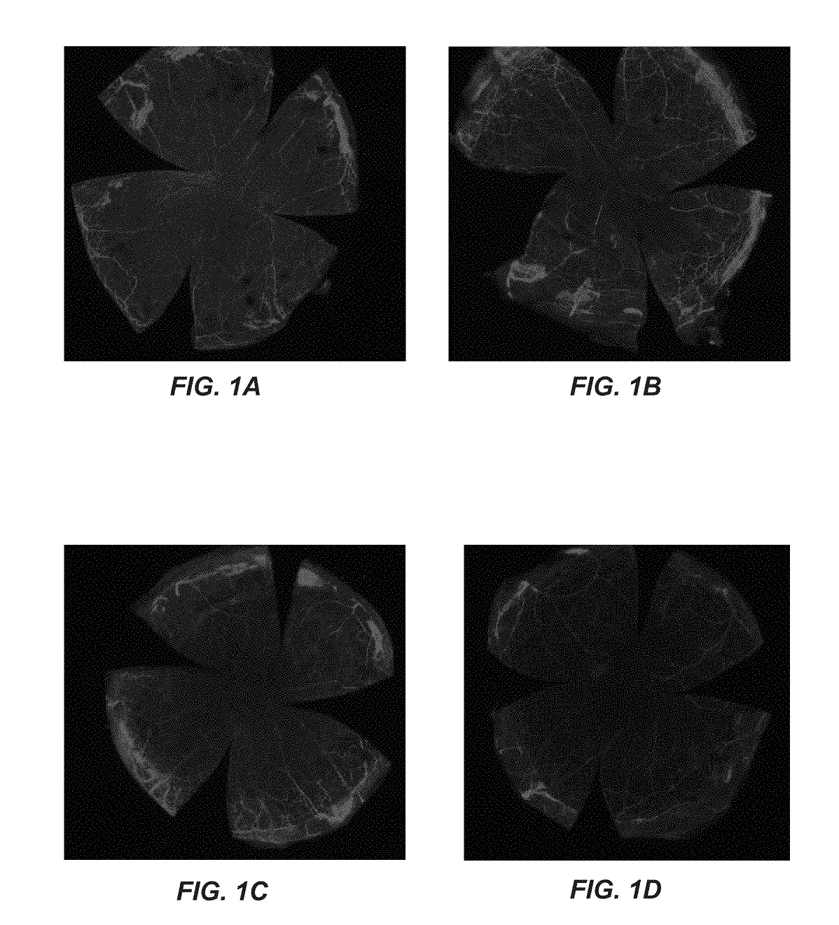

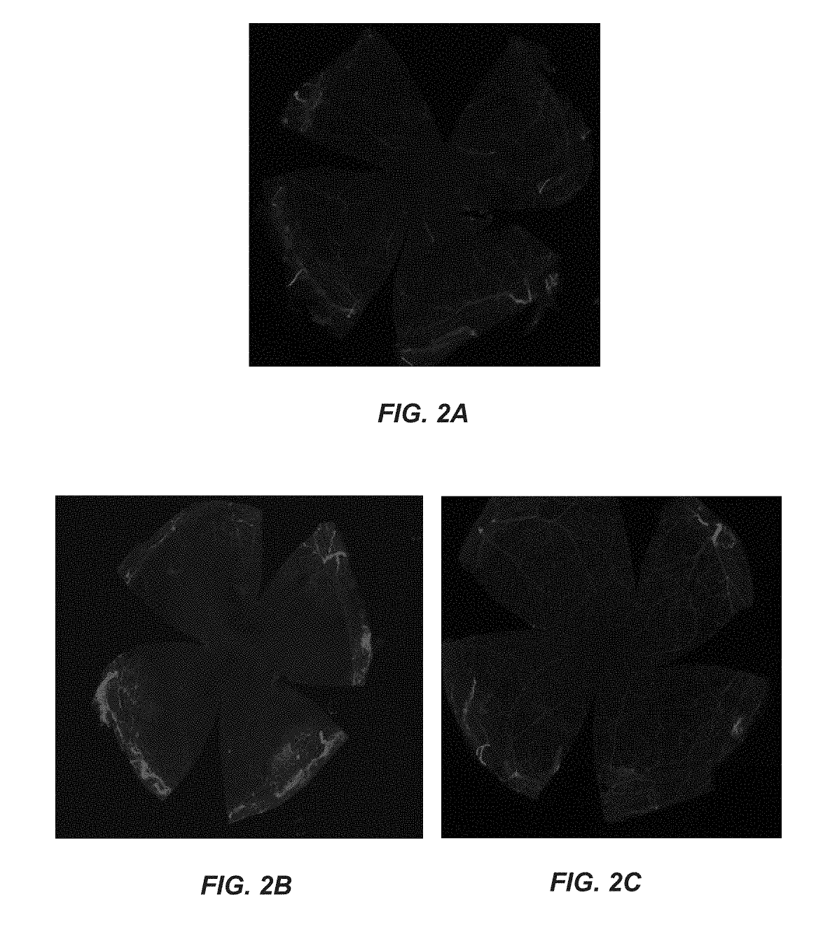

As can be seen in the cornea flat mounts illustrated in FIGS. 1A (normal cornea), 1B (blank treated), 1C (pergolide treated), and 1D (cabergoline treated), compared to the blank control group, the pergolide treated group had improved cornea neuron regeneration. However, the cabergoline treated group showed no neuron regeneration. Based on these preliminary results, it does not appear that cabergoline has the same potential to regenerate cornea neuron fibers as does pergolide. Not wishing to be bound by theory, this may be because pergolide is a D1/D2 agonist, while cabergoline is only a D2/weak D1 agonist. Accordingly, pergolide can effectively upregulate NGF synthesis and secretion as compared to cabergoline which cannot. Further, the cornea primarily includes the D1 receptor, which has a stronger response to pergolide treatment. FIGS. 2A (blank treated), 2B (cabergoline treated), and 2C (pergolide treated) represent additional cornea flat mount results that confirm the results demonstrated in FIGS. 1A-1D that pergolide can much more effectively regenerate cornea neuron fibers than cabergoline.

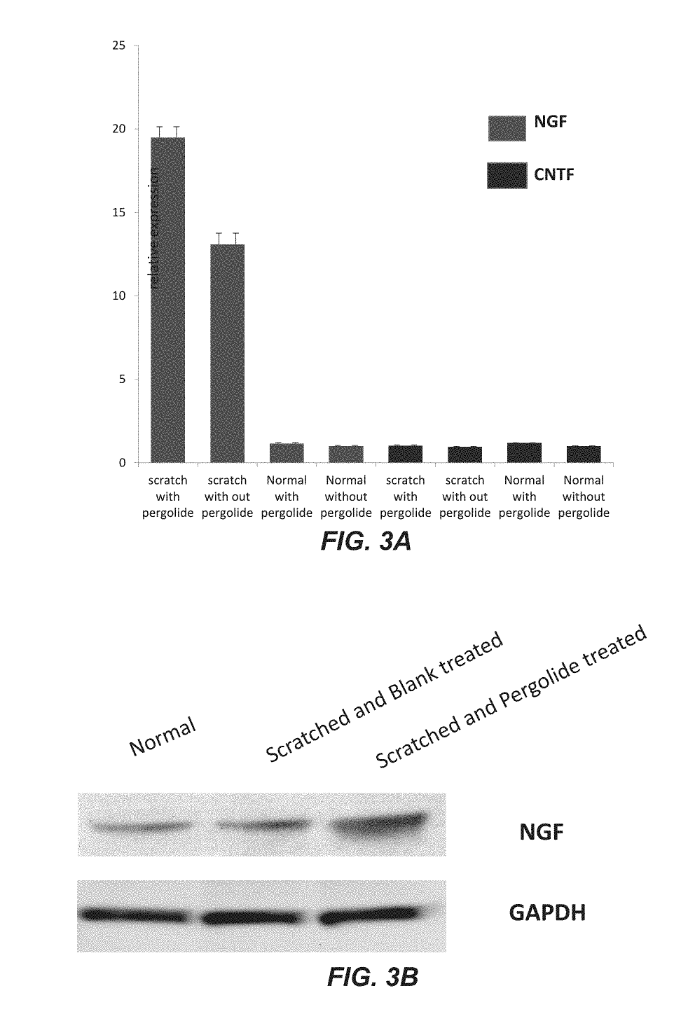

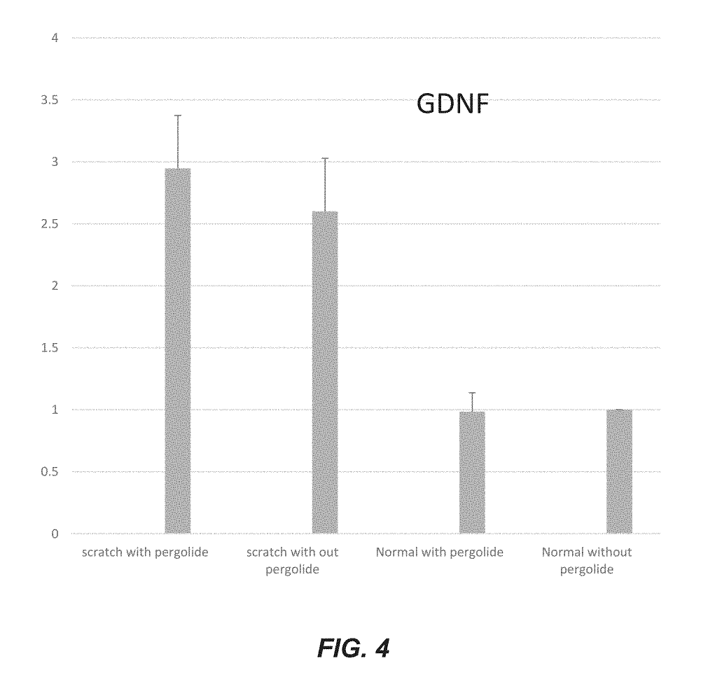

Further, as illustrated in FIGS. 3A and 3B, mice with induced corneal scratches that were treated with pergolide exhibited increased expression of NGF as compared to blank treated mice and normal mice. As illustrated in FIG. 4, pergolide treatment also increased GDNF expression as compared to blank treated and normal mice.

### Example 2

### Pergolide Increases Rate of Corneal Healing

Balb/c mice received induced corneal scratches and were subsequently treated with topical pergolide and blank control eye drops 2 times per day for one week or two weeks. The corneas were then harvested and stained with beta-tubulin antibody. Cornea flatmounts were calculated with ImageJ software.

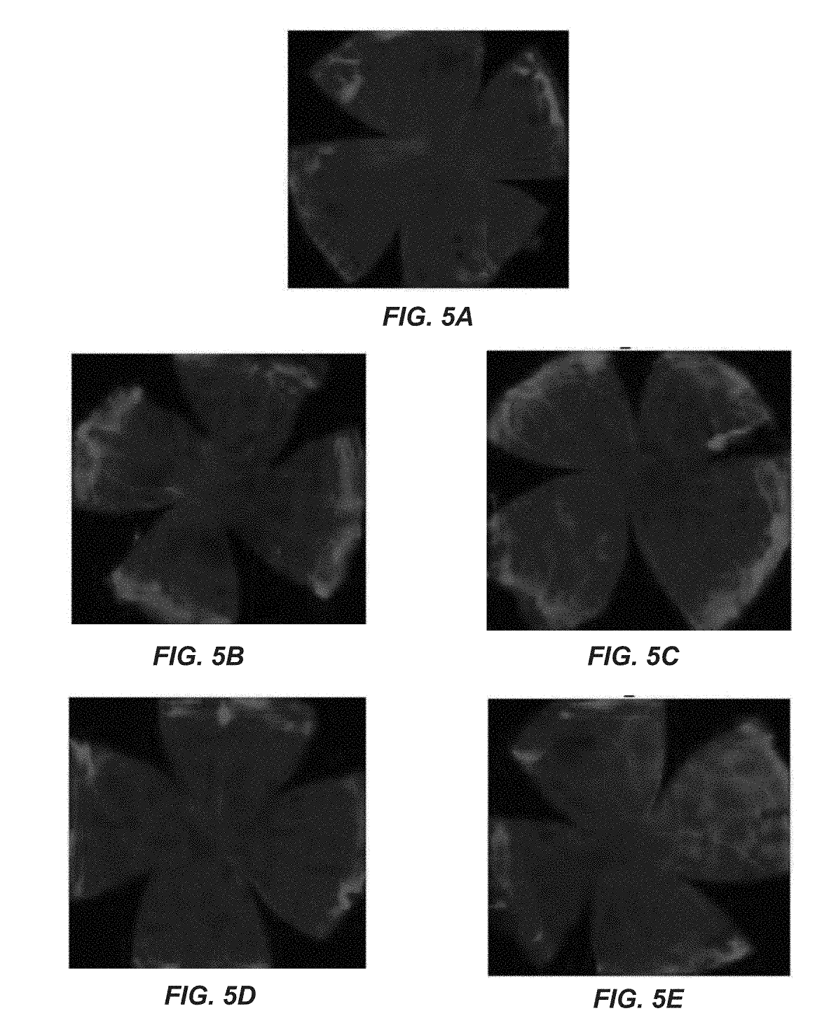

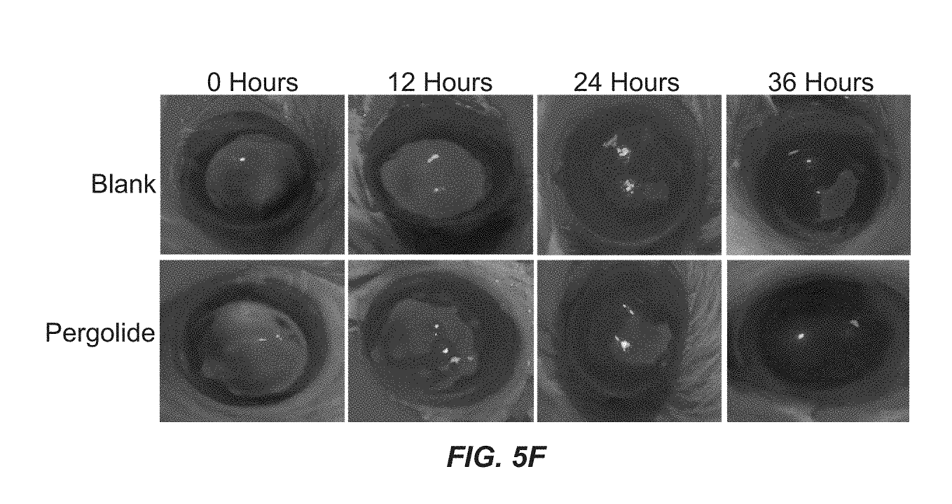

As can be seen in FIGS. 5A (normal), 5B (blank treated, one week), 5C (pergolide treated, one week), 5D (blank treated, two weeks), 5E (pergolide treated, two weeks), and 5F pergolide treated mice exhibited more rapid healing of corneal tissue than blank treated mice after either one week or two weeks of treatment. FIG. 6 illustrates average corneal nerve areas for each of the treatment groups at each time point. This data illustrates that there is no statistical difference between one week and two week treatment periods. Therefore, even a one-week treatment period with pergolide can provide increased healing rates in corneal tissue.

### Example 3

### Pergolide Improves Cornea Nerve Regeneration in STZ-Induced Diabetic Mice

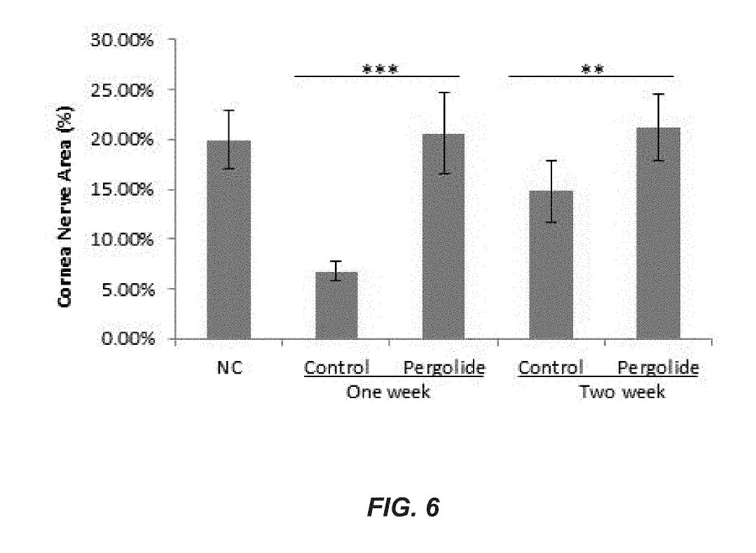

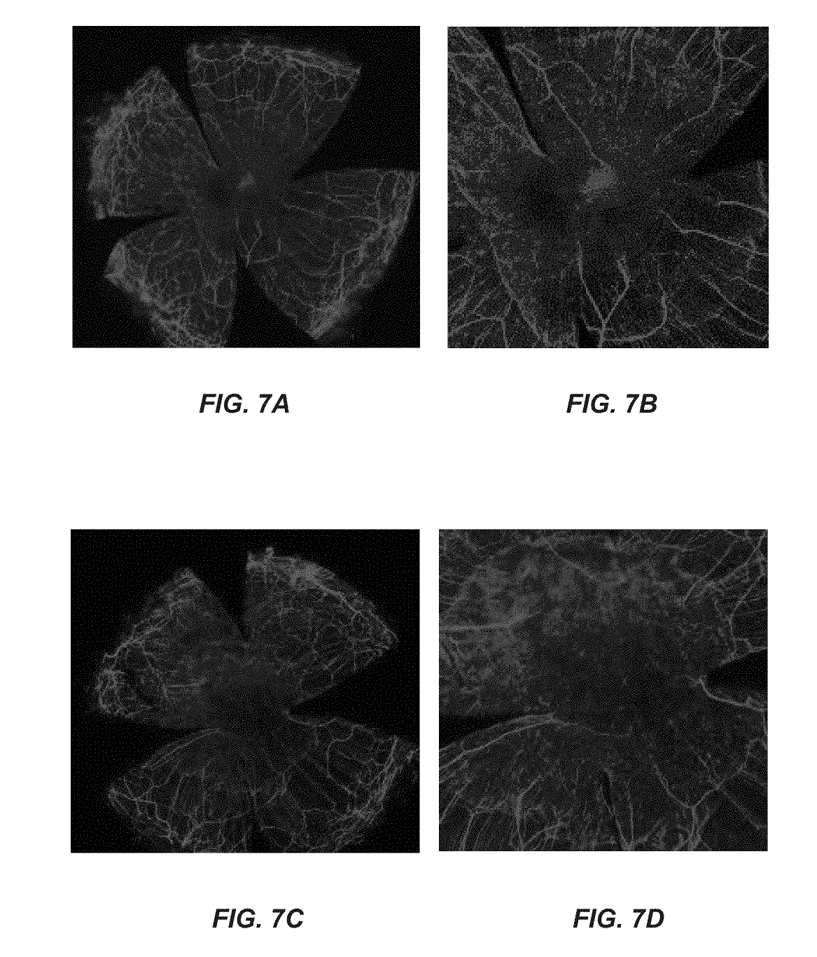

Blank and pergolide eyedrops were administrated on scratched corneas of STZ-induced diabetic mice 3 times per day for 5 days. Cornea flatmounts were harvested 2 days later and images of corresponding flatmounts are illustrated in FIGS. 7A-7D. Beta III tubulin antibody was used to evaluated cornea nerve regeneration. Percentages of corneal nerve area were measured in both the blank treated and pergolide treated subjects. As depicted in FIG. 8, improved corneal nerve regeneration was observed in the pergolide treated mice as compared to the blank treated mice.

### Example 4

### Pergolide Improves Cornea Nerve Regeneration

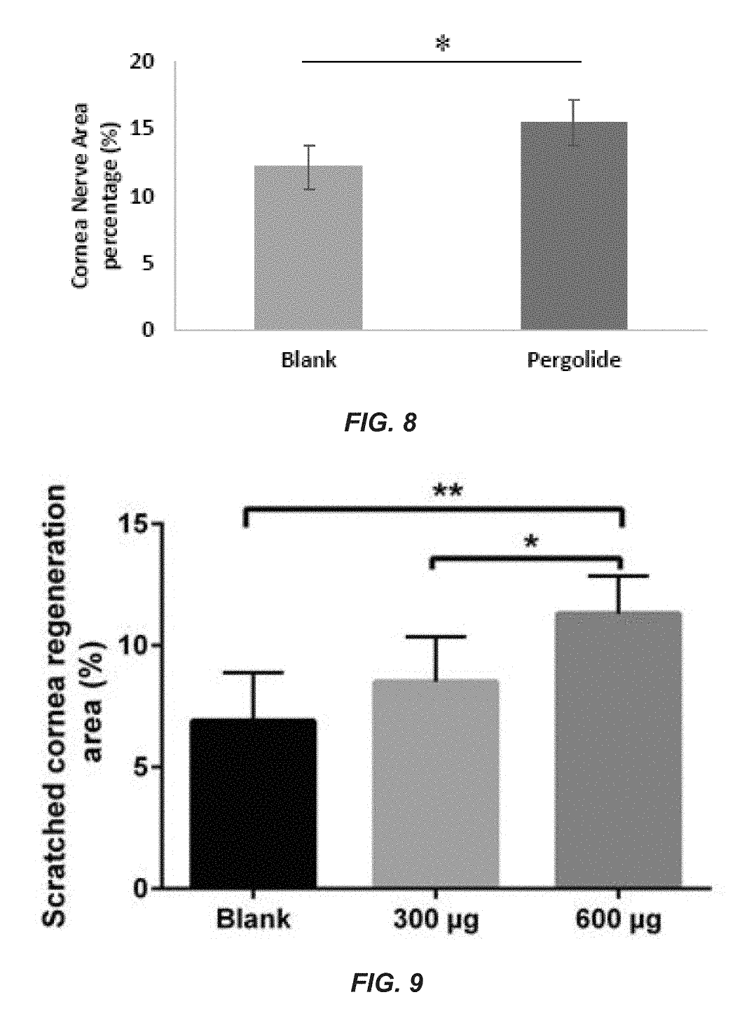

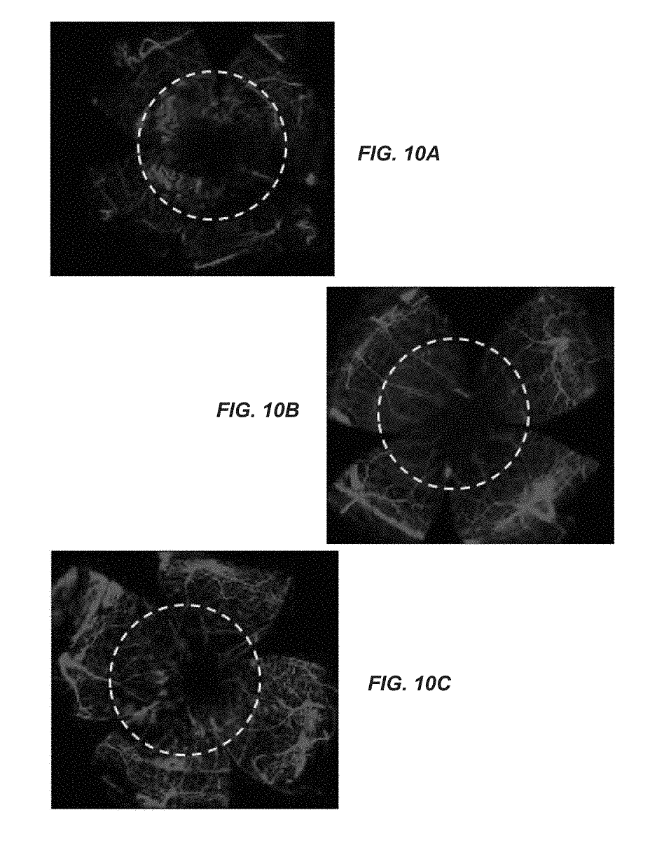

Blank eyedrops and pergolide eyedrops at doses of 300 μg and 600 μg were administrated on scratched corneas of balb/c mice 3 times per day for 1 week. Cornea nerve regeneration was evaluated by Anti-Beta Tubulin III antibody (ab18207, 1:200 in 3% BSA) incubated with the cornea flatmount. As can be seen in FIG. 9, both the 300 μg and 600 μg pergolide doses improved corneal nerve regeneration as compared to the blank eyedrop. Further, the 600 μg pergolide dose showed the greater improvement in corneal nerve regeneration than the 300 μg pergolide dose. It is noted that only that scratch area was calculated, as indicated by the dashed white line in each of FIGS. 10A-10C (FIG. 10A represents the blank-treated sample, FIG. 10B represents the 300 μg pergolide dose-treated sample, and FIG. 10C represents the 600 μg pergolide dose-treated sample). One-way ANOVA was used to analyze statistical difference. * p<0.05; ** p<0.01, n=6.

The following examples pertain to specific technology embodiments and point out specific features, elements, or steps that may be used or otherwise combined in achieving such embodiments.

In one example there is provided, an ophthalmic composition for administration to an eye of a subject, comprising a therapeutically effective amount of pergolide, and pharmaceutically acceptable carrier.

In one example of an ophthalmic composition, wherein the therapeutically effective amount of pergolide is from 0.00005 mg/μl to 0.05 mg/μl.

In one example of an ophthalmic composition, the pharmaceutically acceptable carrier comprises liposomes.

In one example of an ophthalmic composition, at least a portion of the therapeutically effective amount of pergolide is encapsulated or entrapped within the liposomes.

In one example of an ophthalmic composition, the liposomes are formed of a material selected from the group consisting of a phospholipid, cholesterol, a lipid-conjugated hydrophilic polymer, and combinations thereof.

In one example of an ophthalmic composition, the pharmaceutically acceptable carrier comprises a thickening agent, a solubilizing agent, a tonicity agent, a pH adjuster, a preservative, an antioxidant, or a combination thereof.

In one example of an ophthalmic composition, the thickening agent is selected from hyaluronic acid, carboxymethylcellulose, hydroxypropylcellulose, polyvinyl alcohol, polyacrylic acid, xanthan gum, guar gum, dextran, polyvinyl pyrrolidone, polyethylene glycol, and combinations thereof.

In one example of an ophthalmic composition, the solubilizing agent is selected from glycerin, propylene glycol, polyethylene glycol, copolymers of ethylene oxide and propylene glycol, and combinations thereof.

In one example of an ophthalmic composition, the tonicity agent is selected from the group consisting of phosphate-buffered saline (PBS), Alsever's solution, Tris-buffered saline (TBS), water, balanced salt solutions (BSS), sodium chloride, potassium chloride, calcium chloride, magnesium chloride, mannitol, sorbitol, dextrose, glycerin, propylene glycol, ethanol, trehalose, and combinations thereof.

In one example of an ophthalmic composition, the preservative is selected from the group consisting of benzalkonium chloride (BAK), cetrimonium, sodium perborate, ethylenediaminetetraaceticacid (EDTA), chlorobutanol, and combinations thereof.

In one example of an ophthalmic composition, the antioxidant is selected from the group consisting of vitamin E, carnosine, N-acetylcarnosine, pyruvate, resveratrol, astaxanthin, glutathione, cysteine, cysteine ascorbate, and combinations thereof.

In one example of an ophthalmic composition, the composition has a pH of from 5 to 8.

In one example of an ophthalmic composition, the composition has a tonicity of from about 250 to about 350 milliosmoles/liter (mOsm/L).

In one example of an ophthalmic composition, the composition further comprises an additional active agent selected from the group consisting of cabergoline, tacrolimus, glycyl-L-histidyl-L-lysine (GHK), nerve growth factor (NGF), or a combination thereof.

In one example of an ophthalmic composition, the composition is formulated as one of an eye drop, a gel, a thin film, an ointment, or an injectable formulation.

In one example there is provided a method of treating an ophthalmic condition, comprising administering a therapeutically effective amount of pergolide to an eye of a subject in an effective dosage regimen.

In one example of a method of treating an ophthalmic condition, the ophthalmic condition comprises neurotrophic keratopathy, post laser-assisted in situ keratomileusis (LASIK) nerve damage, post radial keratotomy (RK) nerve damage, post penetrating keratoplasty (PK) nerve damage, dry eye, dry age-related macular degeneration (AMID), glaucoma, diabetic retinopathy, retinal degeneration, or combinations thereof.

In one example of a method of treating an ophthalmic condition, administration is performed via intravitreal injection, subconjunctival injection, topical administration, or a combination thereof.

In one example of a method of treating an ophthalmic condition, the effective dosage regimen includes administering an effective amount of pergolide to the eye of the subject from 1 to 3 times per day.

In one example of a method of treating an ophthalmic condition, pergolide is administered as a topical eye drop.

In one example of a method of treating an ophthalmic condition, pergolide is administered in a drop volume of from 10 μl to 60 μl.

In one example of a method of treating an ophthalmic condition, each drop comprises from 0.005 mg to 3 mg of pergolide.

In one example of a method of treating an ophthalmic condition, the effective dosage regimen up-regulates expression of NGF within 48 hours as compared to expression of NGF prior to administration of pergolide.

In one example of a method of treating an ophthalmic condition, the effective dosage regimen up-regulates expression of glial cell-derived neurotrophic factor (GDNF) within 48 hours as compared to expression of GDNF prior to adminstration of pergolide.

While the forgoing examples are illustrative of the principles of the present technology in one or more particular applications, it will be apparent to those of ordinary skill in the art that numerous modifications in form, usage and details of implementation can be made without the exercise of inventive faculty, and without departing from the principles and concepts of the technology.

What is claimed is:

1. An ophthalmic composition for administration to an eye of a subject, comprising:
a therapeutically effective amount of pergolide; and
a pharmaceutically acceptable carrier.

2. The ophthalmic composition of claim 1, wherein the therapeutically effective amount of pergolide is from 0.00005 mg/μl to 0.05 mg/μl.

3. The ophthalmic composition of claim 1, wherein the pharmaceutically acceptable carrier comprises liposomes.

4. The ophthalmic composition of claim 3, wherein at least a portion of the therapeutically effective amount of pergolide is encapsulated or entrapped within the liposomes.

5. The ophthalmic composition of claim 3, wherein the liposomes are formed of a material selected from the group consisting of a phospholipid, cholesterol, a lipid-conjugated hydrophilic polymer, and combinations thereof.

6. The ophthalmic composition of claim 1, wherein the pharmaceutically acceptable carrier comprises a thickening agent, a solubilizing agent, a tonicity agent, a pH adjuster, a preservative, an antioxidant, or a combination thereof.

7. The ophthalmic composition of claim 6, wherein the thickening agent is selected from hyaluronic acid, carboxymethylcellulose, hydroxypropylcellulose, polyvinyl alcohol, polyacrylic acid, xanthan gum, guar gum, dextran, polyvinyl pyrrolidone, polyethylene glycol, and combinations thereof.

8. The ophthalmic composition of claim 6, wherein the solubilizing agent is selected from glycerin, propylene glycol, polyethylene glycol, copolymers of ethylene oxide and propylene glycol, and combinations thereof.

9. The ophthalmic composition of claim 6, wherein the tonicity agent is selected from the group consisting of phosphate-buffered saline (PBS), Alsever's solution, Tris-buffered saline (TBS), water, balanced salt solutions (BSS), sodium chloride, potassium chloride, calcium chloride, magnesium chloride, mannitol, sorbitol, dextrose, glycerin, propylene glycol, ethanol, trehalose, and combinations thereof.

10. The ophthalmic composition of claim 6, wherein the preservative is selected from the group consisting of benzalkonium chloride (BAK), cetrimonium, sodium perborate, ethylenediaminetetraaceticacid (EDTA), chlorobutanol, and combinations thereof.

11. The ophthalmic composition of claim 6, wherein the antioxidant is selected from the group consisting of vitamin E, carnosine, N-acetylcarnosine, pyruvate, resveratrol, astaxanthin, glutathione, cysteine, cysteine ascorbate, and combinations thereof.

12. The ophthalmic composition of claim 1, wherein the composition has a pH of from 5 to 8.

13. The ophthalmic composition of claim 1, wherein the composition has a tonicity of from about 250 to about 350 milliosmoles/liter (mOsm/L).

14. The ophthalmic composition of claim 1, further comprising an additional active agent selected from the group consisting of cabergoline, tacrolimus, glycyl-L-histidyl-L-lysine (GHK), nerve growth factor (NGF), or a combination thereof.

15. The ophthalmic composition of claim 1, wherein the composition is formulated as one of an eye drop, a gel, a thin film, an ointment, or an injectable formulation.

16. A method of treating an ophthalmic condition, comprising:
administering a therapeutically effective amount of pergolide to an eye of a subject in an effective dosage regimen.

17. The method of claim 16, wherein the ophthalmic condition comprises neurotrophic keratopathy, post laser-assisted in situ keratomileusis (LASIK) nerve damage, post radial keratotomy (RK) nerve damage, post penetrating keratoplasty (PK) nerve damage, dry eye, dry age-related macular degeneration (AMD), glaucoma, diabetic retinopathy, retinal degeneration, or combinations thereof.

18. The method of claim 16, wherein the effective dosage regimen includes administering an effective amount of pergolide to the eye of the subject from 1 to 3 times per day.

19. The method of claim 16, wherein pergolide is administered as a topical eye drop.

20. The method of claim 16, wherein the effective dosage regimen up-regulates expression of NGF within 48 hours as compared to expression of NGF prior to administration of pergolide, or upregulates expression of glial cell-derived neurotrophic factor (GDNF) within 48 hours as compared to expression of GDNF prior to adminstration of pergolide, or a combination thereof.
